(19) United States
(12) Patent Application Publication
(10) Pub. No.: US 2026/0347859 A1
(43) Pub. Date: Nov. 5, 2026

(54) IGF-1 RECEPTOR-BINDING APTAMER, AND USE THEREOF

(71) Applicant: POSTECH RESEARCH AND BUSINESS DEVELOPMENT FOUNDATION, Pohang-si (KR)

(72) Inventors: Na Oh YUNN, Pohang-si (KR); Sung Ho RYU, Pohang-si (KR); Seong Eun PARK, Pohang-si (KR); Mangeun PARK, Pohang-si (KR); Eun Ju OH, Pohang-si (KR)

(21) Appl. No.: 19/117,711

(22) PCT Filed: Jul. 20, 2023
(86) PCT No.: PCT/KR2023/010497

§ 371 Date: Apr. 2, 2025

(30) Foreign Application Priority Data
Oct. 14, 2022 (KR) 10-2022-0132552

(51) Int. Cl. C12N 15/115; A61K 31/7115

(52) U.S. Cl. CPC C12N 15/115; A61K 31/7115; C12N 2310/16; C12N 2310/335; C12N 2320/30

(57) ABSTRACT

The present invention relates to an IGF-1 receptor-binding aptamer and a use thereof. The aptamer according to the present invention can function as a biased agonist selectively inducing only the metabolic function of the IGF-1 receptor while promoting phosphorylation of the IGF-1 receptor to enhance glucose uptake. In addition, the aptamer does not promote cell division and thus can alleviate the induction of diabetic retinopathy or cancer development due to excessive cell proliferation caused by IGF-1 administration.

### REFERENCE TO AN ELECTRONIC SEQUENCE LISTING

The contents of the electronic sequence listing (KPO20252146US_SEQ.xml; Size: 25,593 bytes; and Date of Creation: Mar. 13, 2025) is herein incorporated by reference in its entirety. The contents of the electronic sequence listing in no way introduces new matter into the specification.

### TECHNICAL FIELD

The present invention relates to an insulin-like growth factor 1 (IGF-1) receptor-binding aptamer and use thereof.

### BACKGROUND ART

Diabetes is a long-lasting metabolic disease with high blood glucose levels, which can lead to death due to complications such as cardiovascular disease, stroke, and chronic renal failure.

The number of diabetics has been steadily increasing in Korea due to the westernization of diet, and one in seven adults over the age of 30 (14.4%) is currently diagnosed with diabetes, so the demand for diabetes drug development is expected to continue to grow in the future. In particular, injectable insulin preparations have a high share of the diabetes treatment market, accounting for 37%.

Insulin therapy involves the external administration of insulin to individuals with diabetes. It is a crucial treatment of last resort for both of type 1 diabetes, where the body is unable to produce insulin, and type 2 diabetes, which begins with insulin resistance but gradually causes beta cells to lose their function.

Insulin is a hormone that promotes anabolism and activates the synthesis and storage of triglycerides in the liver and adipose tissue. Therefore, insulin administration significantly increases the patient's body weight, which adversely affects the patient's ability to control blood sugar and cardiovascular complications. In addition, insulin easily causes hypoglycemic shock even with a slight overdose because of its very narrow therapeutic index. Hypoglycemia reduces the patient's brain function, causing mental abnormalities and, in severe cases, can lead to coma or death. Despite its importance, the use of insulin therapy is also limited by the two representative side effects mentioned above.

The above-described side effects are due to the innate characteristics of insulin receptors and are difficult to fundamentally resolve, and thus there is currently no clear solution to address the unmet need for side effects of insulin preparations.

However, glycemic control through the IGF-1 receptor has been suggested as one of the various alternatives to this problem. The IGF-1 receptor and insulin receptor are derived from the same ancestral gene and have high homology in protein sequence (about 60% homology) and structure. In terms of physiological functions, both receptors are responsible for regulating metabolism and controlling cell growth and differentiation. Insulin is more active in glucose and lipid metabolism, whereas IGF-1 is more active than insulin in cell division, growth, and differentiation.

Glycemic control via the IGF-1 receptor has several advantages over the insulin receptor. Because the potency and efficacy of increasing glucose uptake through the IGF-1 receptor are lower than those of the insulin receptor, administration of IGF-1 rarely causes hypoglycemic shock induced by insulin administration. Further, IGF-1 receptor is expressed only in muscle and is not present in liver and adipose tissue, and thus administration of IGF-1 selectively increases glucose uptake by muscle and does not affect fat synthesis and storage. Therefore, administration of IGF-1 does not increase body weight.

In particular, clinical trials in which IGF-1 was administered to patients with type 1 and type 2 diabetes have shown significant reductions in blood glucose and decreases in blood insulin concentrations comparable to those seen with insulin. In the short term, IGF-1's ability to control blood sugar was sufficiently effective to replace insulin, and few of the side effects of insulin therapy were observed.

However, clinical trials that attempted to utilize IGF-1 as a diabetes treatment ultimately failed. This is due to IGF-1's excessive promotion of cell division. IGF-1 is a powerful growth factor that greatly promotes cell division, growth, and differentiation. In clinical trials of IGF-1, cases of worsening diabetic retinopathy were observed. It is assumed that this is due to the abnormal proliferation of retinal blood vessels caused by IGF-1 administration. In addition, the safety of IGF-1 in terms of increased cancer incidence with long-term administration is not established. IGF-1 receptor has been shown to play a role in the development of cancers such as breast, lung, and ovarian cancer.

Therefore, the present inventor developed an IGF-1 receptor aptamer as a biased agonist, which is a ligand that selectively activates only a specific function or signal transduction pathway of the IGF-1 receptor, confirmed that the aptamer selectively activates a metabolic function (glucose uptake) but does not promote cell division, thus determined that the IGF-1 receptor aptamer could solve the side effects of IGF-1 and the stability problem for long-term administration of IGF-1 that have been shown in conventional clinical trials, and completed the present invention.

### DISCLOSURE

### Technical Problem

An object of the present invention is to provide an insulin-like growth factor 1 (IGF-1) receptor aptamer that specifically binds to an IGF-1 receptor and promotes phosphorylation of the IGF-1 receptor.

Another object of the present invention is to provide an IGF-1 receptor agonist comprising the IGF-1 receptor aptamer.

Still another object of the present invention is to provide a pharmaceutical composition for preventing or treating a metabolic disease or cancer, comprising the IGF-1 receptor aptamer as an active ingredient.

Still another object of the present invention is to provide a composition for diagnosing diabetes or diabetic complications, comprising the IGF-1 receptor aptamer as an active ingredient.

### Technical Solution

In one general aspect, there is provided an insulin-like growth factor 1 (IGF-1) receptor aptamer that specifically binds to an IGF-1 receptor and promotes phosphorylation of the IGF-1 receptor.

More specifically, there is provided an insulin-like growth factor 1 (IGF-1) receptor aptamer that specifically binds to an IGF-1 receptor, comprising SEQ ID NO: 1 or SEQ ID NO: 2.

The term “aptamer” is a single-stranded oligonucleotide with the length of 15-40 nucleotides that form a specific three-dimensional conformation, has a stem loop structure, and binds specifically to a particular molecule. The aptamer is a compound that is easily synthesized chemically, readily modifiable, heat-stable, and exhibits very high specificity for its target.

Aptamer sequences may be discovered by a method known as selective evolution of ligands by exponential enrichment (SELEX), and hundreds of aptamer sequences are already publicly available. The aptamers are often compared to antibodies in that they specifically bind to targets thereof, but without the immune response. Many aptamers have consistently been discovered with the capability to bind to a variety of target molecules, including small-molecule organic compounds, peptides, and even membrane proteins. Aptamers are often compared to monoclonal antibodies because of their inherent high affinity (usually at the pM level) and specificity to bind to their target molecules, and are specifically referred to as ‘chemical antibodies’, which renders them highly promising as alternative antibodies. The aptamers are very stable compared to antibodies. Unlike protein and antibody drugs, which cannot be stored or transported at room temperature, the aptamers can endure such conditions and maintain their functionality even after sterilization.

As used herein, an IGF-1 receptor aptamer refers to an aptamer capable of binding with specific affinity to an IGF-1 receptor. The IGF-1 receptor may be, but is not limited to, derived from the human IGF-1 receptor protein.

The aptamer is characterized by specifically binding to the IGF-1 receptor and may comprise a modified base in which the nucleotide base at position 5 included in the aptamer is substituted with a hydrophobic functional group.

The bases used in the aptamers of the present invention, other than the above modified bases, are selected from the group consisting of bases A, G, C, T, and deoxy forms thereof, unless otherwise noted, and the inclusion of modifications thereof is specifically noted.

To increase the binding affinity and specificity of the aptamer, a hydrophobic functional group may be substituted at position 5 of the nucleotide base within the aptamer. An example of the substitution may include deoxyribose uracil modified by substituting a hydrophobic functional group at position 5 of the thymine base in the variable region, such as 5-[N-(1-naphthylmethyl)carboxamide]-2′-deoxyuridine (NapdU) or 5-(N-benzylcarboxamide)-2′-deoxyuridine. For example, eight or nine 5-[N-(1-naphthylmethyl)carboxamide]-2′-deoxyuridine (NapdU) or 5-(N-benzylcarboxamide)-2′-deoxyuridine are contained in the variable region. The hydrophobic functional group may comprise a naphthyl group, a benzyl group, a pyrrolebenzyl group, an isobutyl group, or a tryptophan, and more preferably the hydrophobic functional group is a naphthyl group or a benzyl group. Deoxyribose uracil modified by substitution with a naphthyl group is shown in Chemical Formula 1 below, and deoxyribose uracil modified by substitution with a benzyl group is shown in Chemical Formula 2 below:

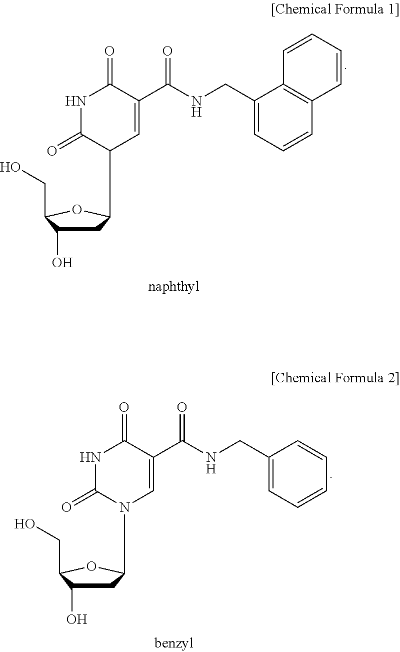

Preferably, the aptamer may comprise SEQ ID NO: 1 or SEQ ID NO: 2. Preferably, the aptamer may be an aptamer that essentially comprises SEQ ID NO: 1 or SEQ ID NO: 2 and additionally comprises consecutive nucleotides on both sides. FIG. 1 shows aptamers having the sequences of SEQ ID NO: 1 (IGF1R-038) and SEQ ID NO: 2 (IGF1R-062) according to the present invention.

TABLE 1

| SEQ ID | | |
|---|---|---|
| NO: | Name | Sequence (5′→3′) |
| 1 | IGF1R-038′ | CGCGAAGGCCANACCGGCNCANGCANNNNAN |
| | | CGAGCG |
| 2 | IGF1R-062′ | CGCGAAGGCCANACCGGCNCANNGCANNNNA |
| | | NCGAGCG |

N: 5-[N-(1-naphthylmethyl)carboxamide]-2′-deoxyuridine or 5-(N-benzyl)carboxamide]-2′-deoxyuridine

In Chemical Formula 1 above, N may be 5-[N-(1-naphthylmethyl)carboxamide]-2′-deoxyuridine or 5-(N-benzyl)carboxamide]-2′-deoxyuridine. In other words, N is a modified base, which may have either a naphthyl group or a benzyl group as a hydrophobic functional group, as referred to in Chemical Formula 1 or 2 above.

Preferably, N is 5-[N-(1-naphthylmethyl)carboxamide]-2′-deoxyuridine, and may be SEQ ID NO: 3 or SEQ ID NO: 4 as shown below.

TABLE 2

| SEQ ID | | |
|---|---|---|
| NO: | Name | Sequence (5′→3′) |
| 3 | IGF1R-038 | CGCGAAGGCCAN′ACCGGCN′CAN′GCA |
| | | N′N′N′N′AN′CGAGCG |
| 4 | IGF1R-062 | CGCGAAGGCCAN′ACCGGCN′CAN′N′ |
| | | GCAN′N′N′N′AN′CGAGCG |

N′: 5-[N-(1-naphthylmethyl)carboxamide]-2′-deoxyuridine

In the above description, C, A, G may be a modified or unmodified base, and may be substituted with a C3 propyl spacer.

For example, the modified C may include a modification of an introduction of a methoxy group (2-O-Me, 2-O-methoxy-DNA) at the C2 position of the nucleotide; or an introduction of fluorine (2-F, 2-fluorine-DNA) at the C2 position of the nucleotide.

Specifically, the modified G may include a modification of an introduction of a methoxy group (2-O-Me, 2-O-methoxy-DNA) at the C2 position of the nucleotide; or an introduction of fluorine (2-F, 2-fluorine-DNA) at the C2 position of the nucleotide.

Specifically, the modified A may include a modification of an introduction of a methoxy group (2-O-Me, 2-O-methoxy-DNA) at the C2 position of the nucleotide; or an introduction of fluorine (2-F, 2-fluorine-DNA) at the C2 position of the nucleotide.

Preferably, the aptamer may form a stem-loop structure comprising nucleotides inside.

The aptamer according to the present invention essentially comprises the sequences of SEQ ID NO: 1 and/or SEQ ID NO: 2 as shown above. More specifically, the aptamer may consist of or essentially consist of, or may comprise SEQ ID NO: 1 and/or SEQ ID NO: 2.

More specifically, the sequence may comprise a sequence having at least 70%, at least 80%, at least 85%, or at least 90% homology.

Further, the sequences may be approximately at least 37, 38, 39, 40, 41, 42, 43, 44, 45, 46, 47, 48, 49, 50 consecutive sequences.

As an example, the sequence may be SEQ ID NO: 5 or 6 as shown below.

TABLE 3

| SEQ ID | | |
|---|---|---|
| NO: | Name | Sequence (5′→3′) |
| 5 | IGF1R-038″ | TATCGAGCGTCCTGCCTTTGAACGCGAAGG |
| | | CCANACCGGCNCANGCANNNNANCGAGCGA |
| | | CACCGACAGCCACCCAGAAA |
| 6 | IGF1R-062″ | TATCGAGCGTCCTGCCTTTGAACGCGAAGG |
| | | CCANACCGGCNCANNGCANNNNANCGAGCG |
| | | ACACCGACAGCCACCCAGAAA |

N: 5-[N-(1-naphthylmethyl)carboxamide]-2′-deoxyuridine or 5-(N-benzyl)carboxamide]-2′-deoxyuridine

In Chemical Formula 1 above, N may be 5-[N-(1-naphthylmethyl)carboxamide]-2′-deoxyuridine or 5-(N-benzyl)carboxamide]-2′-deoxyuridine. In other words, N is a modified base, which may have either a naphthyl group or a benzyl group as a hydrophobic functional group, as referred to in Chemical Formula 1 or 2 above.

As a preferred example, N is 5-[N-(1-naphthylmethyl)carboxamide]-2′-deoxyuridine, and may be SEQ ID NO: 7 or SEQ ID NO: 8 as shown below.

TABLE 4

| SEQ ID | | |
|---|---|---|
| NO: | Name | Sequence (5′→3′) |
| 7 | IGF1R- | TATCGAGCGTCCTGCCTTTGAACGCGAAGGCCA |
| | 038′″ | N′ACCGGCN′CAN′GCAN′N′N′N′AN′CGAGC |
| | | GACACCGACAGCCACCCAGAAA |
| 8 | IGF1R- | TATCGAGCGTCCTGCCTTTGAACGCGAAGGCCA |
| | 062′″ | N′ACCGGCN′CAN′N′GCAN′N′N′N′AN′CGA |
| | | GCGACACCGACAGCCACCCAGAAA |

N′: 5-[N-(1-naphthylmethyl)carboxamide]-2′-deoxyuridine

In the present invention, comprising a sequence may mean including a sequence, may mean essentially consisting of a sequence, or may mean consisting of a sequence.

Furthermore, in any sequence other than the position of N in SEQ ID NO: 1 and/or SEQ ID NO: 2, at least one base located at the 5′ end, 3′ end, middle or both ends may be modified to enhance stability in serum or to modulate renal clearance. The modification may be achieved by one or more selected from the group consisting of polyethylene glycol (PEG), biotin, inverted deoxythymidine (idT), locked nucleic acid (LNA), 2′-methoxy nucleoside, 2′-amino nucleoside, 2′F-nucleoside, amine linker, thiol linker, and cholesterol, bound at the 5′ end, 3′ end, middle, or both ends. The 2′-methoxy nucleoside, 2′-amino nucleoside, or 2′F-nucleoside binds to a base within the aptamer to form a modified base, thereby providing nuclease resistance.

Preferably, a modified base may be further included to secure nuclease resistance and provide stability when administered in humans. The modified base may preferably be a 2′-OMe (methoxy) and/or 2′-F (fluorine) substitution on any of the A, C, T, or G bases. In other words, at least one modified base substitution of 2′-OMe (methoxy) or 2′-F (fluorine) on the base of A, C, T, or G of SEQ ID NO: 1 and/or SEQ ID NO: 2 above, may be included.

The present inventors performed SELEX using IGF-1 receptor protein and newly selected aptamers that bind more strongly to the IGF-1 receptor. The two aptamers selected in this manner are the IGF1R-038 (SEQ ID NO: 3) and IGF1R-062 aptamers (SEQ ID NO: 4) described above.

The aptamers according to the present invention may promote phosphorylation of the IGF-1 receptor, and specifically, preferentially phosphorylate tyrosine at position 1135 (Y1135) in the amino acid sequence of the IGF-1 receptor and then promote phosphorylation of serine at position 473 of AKT (AKT S473), thereby enabling glucose uptake.

Additionally, unlike IGF-1, the aptamers according to the present invention do not promote cell division.

Unlike insulin administration, IGF-1 rarely causes hypoglycemic shock and does not increase body weight. However, IGF-1 is a powerful growth factor that significantly promotes cell division, growth, and differentiation, and thus cases have been observed to worsen diabetic retinopathy, and are known to play an important role in the development of cancers such as breast cancer, lung cancer, and ovarian cancer.

The present inventors have confirmed through Examples that the aptamers increase cellular glucose uptake to a degree comparable to insulin while not promoting cell division, and therefore, the aptamers according to the present invention may be usefully employed for regulating blood glucose levels without exhibiting the side effects of existing insulin and IGF-1.

Two or more aptamers according to the present invention may be linked to exist as a dimer or multimer.

The formation of a dimer or multimer may be implemented by binding two or more aptamers via the complementary nucleic acid sequences to achieve a dimer or multimer form and/or by binding two or more aptamers via the spacer sequence to achieve a single-stranded form.

A “linker sequence” refers to a chemical moiety that connects two molecules or moieties. The linker sequences of the present invention correspond to, for example, nucleic acid linkers, which form double bonds through their complementary base sequences to form dimers. Further, two or more linker sequences may be included as needed to form multimers.

The linker sequence having the complementary sequence may have, for example, 1, 2, 3, 4, 5, 6, 7, 8, 9, 10, 15, 20, 25, 30, 40, 50, 60 sequences, preferably have 5 to 30 base sequences, 10 to 30 base sequences, approximately 15 to 25 base sequences, and more preferably about 20 base sequences.

The linker sequence may further comprise a modified base to secure nuclease resistance and provide stability when administered in human. The modified base may preferably be a 2′-OMe (methoxy) and/or 2′-F (fluorine) substitution on any base A, C, T, or G, and may be L-form DNA or LNA (Locked DNA). In other words, the sequence of the linker sequence may have at least one 2′-OMe (methoxy) or 2′-F (fluorine) modified base substitution on the base A, C, T, or G, and may utilize a form of L-form DNA or Locked DNA (LNA) within the linker sequence.

Further, the linker sequence may further comprise a spacer sequence to provide appropriate spacing between the linker sequence and the sequence of SEQ ID No: 1 that binds to the insulin receptor, if desired. The sequence for providing space between the linker and the insulin receptor to form the complementary bond is preferably polyT and may be included between the linker and the insulin receptor in approximately 0, 1, 2, 3, 4, 5, or 6 fragments.

As the spacer sequence, poly A fragments, poly T fragments, poly G fragments, poly C fragments, phosphoramidite fragments, random oligonucleotide sequences that do not interfere with the aptamer sequence, ethylene glycol fragments, and propyl fragments may be utilized. The length of this fragment may vary as is known to those skilled in the art. Preferably, the spacer sequence may be 2, 3, 4, 5, 6, 7, 8, 9, 10, 11, 12, 13, 14, 15, 16, 17, 18, 19, 20, 21, 22, 23, 24, 25, 26, 27, 28, 29, 30 poly T fragment sequences.

The spacer sequence may further comprise a modified base to secure nuclease resistance and provide stability when administered in humans. The modified base may preferably be a 3′-propyl spacer, 2′-OMe (methoxy) and/or 2′-F (fluorine) substitution on any base A, C, T, or G, and may be L-form DNA or LNA (Locked DNA). In other words, the sequence of the linker sequence may have at least one 2′-OMe (methoxy) or 2′-F (fluorine) modified base substitution on the base A, C, T, or G, and may be in the form of L-form DNA or Locked DNA (LNA) within the linker sequence.

The present invention provides an IGF-1 receptor agonist comprising the aptamer as described above.

The term “IGF-1 receptor agonist” in the present invention refers to a pharmaceutically acceptable agent that selectively binds to an IGF-1 receptor. In general, the IGF-1 receptor agonist refers to a new type of antidiabetic agent developed to effectively control blood glucose. The IGF-1 receptor agonist of the present invention is characterized by its specific binding to the IGF-1 receptor and the absence of side effects such as diabetic retinopathy and cancer development.

Therefore, the IGF-1 receptor agonist may be used for the diagnosis or treatment of various diseases related to insulin, especially metabolic diseases.

Accordingly, in another aspect, the present invention provides a pharmaceutical composition for the prevention or treatment of metabolic diseases or cancer, comprising the aptamer as described above.

For example, the metabolic disease may be diabetes (T1D and/or T2DM, e.g., prediabetes), idiopathic type 1 diabetes (T1D (type 1b)), latent autoimmune diabetes in adults (LADA), early-onset type 2 diabetes (T2DM (EOD)), juvenile-onset atypical diabetes (YOAD), adult-onset diabetes of the young (MODY), malnutrition-related diabetes, gestational diabetes, hyperglycemia, insulin resistance, hepatic insulin resistance, impaired glucose tolerance, diabetic neuropathy, diabetic nephropathy, renal disease (e.g., acute renal failure, tubular dysfunction, inflammatory changes to proximal tubules), diabetic retinopathy, adipocyte dysfunction, visceral fat accumulation, sleep apnea, obesity (e.g., hypothalamic obesity and monogenic obesity) and related comorbidities (e.g., osteoarthritis and urinary incontinence), eating disorders (e.g., binge eating syndrome, bulimia nervosa, and syndromic obesity, such as Prader-Willi syndrome and Bardet-Biedl syndrome), weight gain due to use of other medications (e.g., from use of steroids and antipsychotics), excessive sugar cravings, dyslipidemia (including hyperlipidemia, hypertriglyceridemia, increased total cholesterol, high LDL cholesterol, and low HDL cholesterol), or nonalcoholic fatty liver disease (NAFLD) (including related conditions such as steatosis, nonalcoholic steatohepatitis (NASH), fibrosis, cirrhosis, and hepatocellular carcinoma).

Specifically, the cancer may be any one selected from the group consisting of Ewing sarcoma, rhabdomyosarcoma, multiple myeloma, lymphoma, leukemia, breast cancer, lung cancer, prostate cancer, pancreatic cancer, rectal cancer, colon cancer, colorectal cancer, cervical cancer, and bile duct cancer.

In particular, the aptamer of the present invention inhibits the activity (expression) of IGF-1 receptor (IGF-1R), thereby imparting an anticancer effect to cancer with high expression of IGF receptor.

More specifically, it may be a cancer with high expression of IGF receptor. The cancers with high expression of IGF receptor may be any one selected from the group consisting of Ewing sarcoma, rhabdomyosarcoma, multiple myeloma, lymphoma, leukemia, breast cancer, lung cancer, prostate cancer, pancreatic cancer, rectal cancer, colon cancer, colorectal cancer, cervical cancer, and bile duct cancer.

As used herein, the “pharmaceutically effective amount” means an amount sufficient to treat a disease at a reasonable benefit/risk ratio applicable to medical treatment, and the effective dose level may be determined depending on factors including the patient's gender, age, type and severity of the disease, activity of the drug, sensitivity to the drug, time of administration, route of administration and excretion rate, duration of treatment, concurrently used drugs, and other factors well known in the medical field.

The pharmaceutical composition of the present invention may be administered as an individual therapeutic agent or in combination with other therapeutic agents, or may be administered sequentially or simultaneously with conventional therapeutic agents. Further, the pharmaceutical composition of the present invention may be administered as a single dose or in multiple doses. In consideration of all of the above factors, it is important to administer an amount capable of obtaining the maximum effect with the minimum amount without side effects, which may be easily determined by those skilled in the art.

More preferably, the pharmaceutical composition according to the present invention may be administered alone or in combination with insulin. The pharmaceutical composition according to the present invention may be used in combination with or in place of insulin to reduce the amount of insulin used, thereby reducing the side effects experienced by patients with severe diabetes.

As used herein, the term “subject” includes an animal or human whose symptoms may be improved by the administration of the pharmaceutical composition according to the present invention. By administering the composition for treatment according to the present invention to an individual, metabolic diseases or cancer may be effectively prevented and treated.

As used herein, the term “administration” means introducing a predetermined substance into humans or non-human animals by any appropriate method, and the administration route of the composition for treatment of the present invention may be oral or parenteral through any general route as long as the composition is possible to reach the target tissue. Further, the composition for treatment of the present invention may be administered by any device capable of delivering an active ingredient to a target cell.

The preferred dosage of the pharmaceutical composition according to the present invention depends on the condition and weight of the patient, the severity of the disease, the form of the drug, the route of administration and the duration, but may be suitably selected by those skilled in the art.

The pharmaceutical composition may be formulated in various oral dosage forms or parenteral dosage forms. For example, the composition may be in any formulation for oral administration, such as tablets, pills, hard and soft capsules, liquids, suspensions, emulsions, syrups, granules, elixirs, and the like. Such formulation for oral administration may contain, in addition to the active ingredient, pharmaceutically acceptable carriers, such as diluents such as lactose, dextrose, sucrose, mannitol, sorbitol, cellulose and/or glycine, or lubricants such as silica, talc, stearic acid, and magnesium or calcium salts thereof and/or polyethylene glycol, depending on the usual composition of each formulation.

Further, the formulation for oral administration may comprise, in the case of a tablet, a binder such as magnesium aluminum silicate, starch paste, gelatin, tragacanth, methylcellulose, sodium carboxymethylcellulose and/or polyvinylpyrrolidine, and in some cases, may comprise a disintegrating agent such as starch, agar, alginic acid or sodium salts thereof, or a boiling mixture and/or absorbent, colorant, flavor or sweetener.

When provided for parenteral use, the formulation may be, for example, in the form of liquid, gel, cleansing composition, tablet for insertion, suppository form, cream, ointment, dressing solution, spray, or other topical application, liquid formulations such as a solution, a suspension, an emulsion, and the like, and may comprise an external skin preparation such as a sterilized aqueous solution, non-aqueous solvent, suspension, emulsion, freeze-dried formulation, suppository, cream, ointment, jelly, foam, detergent or insert, preferably a liquid or gel, cleansing composition, and a tablet for insertion, etc. The formulation may be prepared, for example, by adding a solubilizing agent, emulsifier, buffer for pH adjustment, etc., to sterile water. As the non-aqueous solvent and the suspension, propylene glycol, polyethylene glycol, vegetable oils such as olive oil, and injectable esters such as ethyl oleate, etc., may be used.

As needed, the aptamer may be entrapped within a colloidal drug delivery system (such as liposome, albumin microsphere, microemulsion, nanoparticle, and nanocapsule) or within microcapsules prepared by coacervation techniques or interfacial polymerization, such as hydroxymethylcellulose or gelatin-microcapsules and poly-(methylmethacrylate) microcapsules, respectively. These techniques are known in the relevant art.

The pharmaceutical composition according to the present invention may be administered in a pharmaceutically effective amount. As used herein, the “pharmaceutically effective amount” means an amount sufficient to prevent or treat the diseases at a reasonable benefit/risk ratio applicable to medical treatment. The effective dose level may be varied by those skilled in the art depending on factors such as method of formulation, patient condition and weight, patient's gender, age, severity of disease, drug form, route and duration of administration, rate of excretion, response sensitivity, etc. The effective amount may vary depending on the route of processing, the use of excipients and the possibility of use with other medicaments, as recognized by those skilled in the art. However, for desirable effects, in the case of oral administration, the composition of the present invention may be administered to an adult at an amount of 0.0001 to 100 mg/kg of body weight per day, preferably 0.001 to 100 mg/kg per day, but the above dosages are not intended to limit the scope of the present invention in any way.

Further, the pharmaceutical composition may be formulated in a parenteral dosage form, in which case it is administered by a parenteral route, such as subcutaneous injection, intravenous injection, intramuscular injection, or intrathoracic injection. Here, for preparation into a dosage form for parenteral administration, the pharmaceutical composition may be prepared as a solution or suspension in which the active ingredient, i.e., a derivative of Chemical Formula I or a pharmaceutically acceptable salt thereof, is mixed in water with a stabilizer or buffer, and this solution or suspension may be prepared in unit dosage form in ampoules or vials.

Further, the pharmaceutical composition may be sterile, may further comprise an auxiliary such as preservative, stabilizer, hydrating agent or emulsification promoter, salt and/or buffer for osmotic pressure control, or may further comprise other therapeutically useful substances, and may be formulated according to conventional methods of mixing, granulating, or coating.

The pharmaceutical composition of the present invention may be administered to a mammal, preferably a rodent or a human.

Meanwhile, since the aptamer according to the present invention may effectively absorb glucose, in another aspect, the present invention may additionally provide a hypoglycemic agent comprising the above-described aptamer as an active ingredient.

Here, more preferably, the hypoglycemic agent may be administered via subcutaneous injection to provide an immediate effect.

The present invention provides a method for preventing or treating a metabolic disease or cancer, comprising administering the above-described aptamer to a subject in need thereof.

The present invention provides a composition for preventing or treating a metabolic disease or cancer, comprising the aptamer as described above.

The present invention provides use of the aptamer as described above in the manufacture of a medicament for the treatment of a metabolic disease or cancer.

In another aspect, the present invention provides a composition for diagnosing diabetes or diabetic complications, comprising the aptamer as described above as an active ingredient.

The diagnostic composition according to the present invention may be used by reacting with a biological sample. The corresponding reaction results in the binding of the aptamer according to the present invention to the IGF-1 receptor in the biological sample, wherein if the binding degree of the aptamer according to the present invention is higher than that of a normal sample, the biological sample may be diagnosed as having diabetes or diabetes complications.

The binding degree of the aptamer according to the present invention in the biological sample may be determined using DNA aptamer binding measurement techniques conventionally used in the art, such as, but not limited to, labeling the ends of the aptamer with a fluorescent or radioactive substance and measuring the fluorescence or radioactivity intensity, or imaging and observing the fluorescence or radioactivity.

### Advantageous Effects

The aptamer according to the present invention can function as a biased agonist selectively inducing only the metabolic function of the IGF-1 receptor while promoting phosphorylation of the IGF-1 receptor to enhance glucose uptake. In addition, the aptamer does not promote cell division and thus can alleviate the induction of diabetic retinopathy or cancer development due to excessive cell proliferation caused by IGF-1 administration.

### DESCRIPTION OF DRAWINGS

FIG. 1 shows the sequences of IGF1R-038 and IGF1R-062 aptamers according to the present invention.

FIG. 2 shows the results of the phosphorylation of IGF-1 receptor by IGF1R-038 and IGF1R-062 aptamers according to the present invention.

FIG. 3 shows the results of the phosphorylation of IGF-1 receptor, AKT and ERK by IGF1R-038 aptamer according to the present invention.

FIG. 4 shows the inhibition of IGF-1 activity by the IGF1R-038 aptamer according to the present invention.

FIG. 5 shows the concentration-dependent glucose uptake of the IGF1R-038 aptamer according to the present invention.

FIG. 6 shows the effect on cell growth when IGF-1 and IGF1R-038 according to the present invention were treated alone in MCF-7 cells.

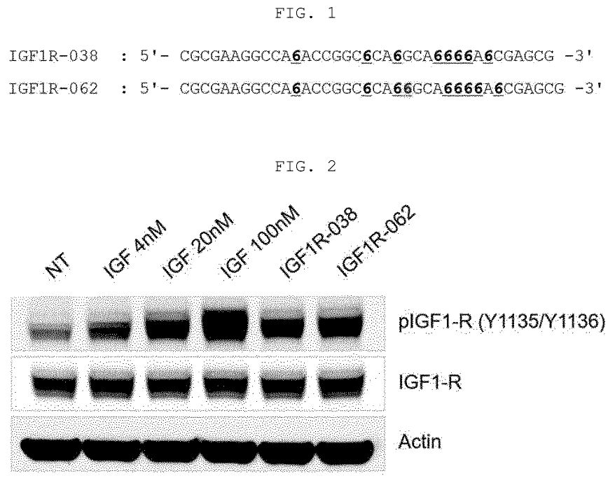

### BEST MODE

The following exemplary embodiments are presented to help understanding of the present invention. These Examples and Experimental Examples are only provided to more easily understand the present invention, but the content of the present invention is not limited by these Examples.

### Preparation of Antibodies and Reagents

Human IGF-1 receptor protein was purchased from R&D system (Minneapolis, MN). The aptamer used was synthesized by Aptamer Sciences, Inc. The antibodies used in the Western blot were as follows: Anti-phosphor-IGF-1R (Y1135), Anti-IGF-1R, Anti-phosphor-ERK (T202/Y204), Anti-phosphor-AKT (S473), Anti-phosphor-AKT (T308), and Anti-actin were purchased from Cell signaling (Beverly, MA). Anti-phosphor-IGF-1R (Y1135/Y1136) was purchased from Invitrogen (Carlsbad, CA).

### Cell Culture

CHO cells, MCF-7 cells, and L6 myoblast cells were purchased from the American Type Culture Collection (Manassas, VA, USA). 293F cells were purchased from Thermo Scientific Inc. MCF-7, 293F, and Rat-1/hIR were cultured in DMEM (Dulbecco's modified Eagle's medium, Lonza) supplemented with 10% fetal bovine serum (FBS, Gibco), 100 units/ml penicillin and 100 units/ml streptomycin at 37 °C and 5% CO₂. L6 myoblast cells were cultured in Alpha MEM (WELGEN) supplemented with 10% bovine serum (Gibco), 100 units/ml penicillin and 100 units/ml streptomycin at 37 °C and 5% CO₂.

### Example 1. IGF-1 Receptor Aptamer Screening

SELEX (Systematic Evolution of Ligands by Exponential Enrichment) was used to screen for aptamers that bind to the extracellular domain of the insulin receptor. SELEX was conducted using a single-stranded DNA library (SEQ ID NO: 3) consisting of a 40-mer variable sequence flanked by 20-mer constant sequences on both sides.

### 1.1 Synthesis of Modified Nucleic Acid Library

To construct the single-strand modified DNA library required for SELEX, an antisense library (SEQ ID NO: 3) with biotin attached at the 5′ end was synthesized. The antisense library was reacted with 50 μM reverse primer (SEQ ID NO: 4) in 0.5 mM dNTP (ATP, GTP, CTP, Bz-dU), 0.25 U/μl KOD XL (Invitrogen), 10× extension buffer (1.2 M Tris-HCl pH 7.8, 100 mM KCl, 60 mM (NH₄)₂SO₄, 70 mM MgSO₄, 1% TritonX-100, 1 mg/ml BSA) at 70 °C for 1 hour to produce double-stranded DNA. The single-stranded modified DNA library was eluted using 20 mM NaOH and neutralized with HCL solution. The prepared DNA library was concentrated using Amicon ultra-15 (Millipore) and quantified using a UV spectrophotometer.

### 1.2 Binding to IGF-1 Receptor

1 nM of the above synthesized library was added to selection buffer (200 mM HEPES, 510 mM NaCl, 25 mM KCl, and 25 mM MgCl₂) and reacted at 95 °C, 70 °C, 48 °C, and 37 °C for 5 minutes each. Then, for negative selection, 10 μL of 10× protein competition buffer (10 μM prothrombin, 10 μM casein, 0.1% (w/v) human serum albumin (HSA, SIGMA)) was mixed and added to Dynabeads® MyOne™ Streptavidin C1 (SA beads) (50% (v/v) slurry, 10 mg/mL Invitrogen) after removing the supernatant and the obtained mixture was reacted at 37 °C for 10 minutes.

After the negative selection reaction, only the supernatant was taken, transferred to a new tube, and reacted with Dynabead TALON conjugated with His-tagged IGF-1 receptor protein at 37 °C for 1 hour. Dynabeads TALON bound to DNA and IGF-1 receptor were washed five times with 100 μl of selection buffer (200 mM HEPES, 510 mM NaCl, 25 mM KCl, 25 mM MgCl₂). At the fifth wash, the libraries were transferred to a fresh plate and washed. The library binding to the target was eluted by adding 85 μl of 2 mM NaOH solution, followed by neutralization with 20 μl of 8 mM HCl solution.

### 1.3 Amplification

The library DNA binding to the target was amplified using quantitative PCR (QPCR, IQ5 multicolor real time PCR detection system, Bio-rad). The reverse primer (SEQ ID NO: 4) and antisense library (SEQ ID NO: 3) used previously for library preparation were amplified using 5 μM (5×QPCR master Mix, Novagen), 0.075 U/μl KOD (Novagen), 1 mM dNTPs (Roche Applied science), 25 mM MgCl₂, and 5× SYBRgreen I (Invitrogen) were mixed to a total volume of 125 μM, and the mixture was subjected to the following conditions: 1 cycle at 96 °C for 15 seconds, 55 °C for 10 seconds, and 68 °C for 30 minutes, followed by 30 cycles of 96 °C for 15 seconds and 72 °C for 1 minute to generate the double-stranded library.

1.4 eDNA Synthesis

The term eDNA is enzymatic DNA, which means aptamers produced using DNA template and polymerase. The DNA library generated by the above QPCR was immobilized on 25 μl Myone SA beads (Invitrogen) by mixing for 10 minutes at room temperature. Here, the amount of mixed DNA was 60 μl as QPCR product. The single-stranded DNA was obtained by adding 20 mM NaOH solution. Then, DNA containing the modified nucleic acid was synthesized in the same way as the library synthesis in Example 1.1 and used in the next round. A total of 8 SELEX rounds were performed, and for more selective binding, DNA aptamers were screened by diluting the DNA and protein (IntegrinαVβ3) complexes by 1/200 and 1/400 in 10 mM DxSO4 (sigma) solution from round 4 to 6 and from round 7 to 8, respectively.

### 1.5 Aptamer Sequence Analysis

After 8 rounds of SELEX, the resulting products were amplified into double-stranded DNA using QPCR, and then purified using QIAquick PCR Purification Kit (QIAGEN). DNA sequences were analyzed in 150 paired end read format using Illumina NextSeq500.

### Experimental Example 1. Evaluation of Phosphorylation of IGF-1 Receptor Aptamer

### 1.1 Western Blot

Prior to treatment with aptamers, cells were treated with FBS-free DMEM for 3 hours for serum starvation. All aptamers were heated at 95 °C for 5 minutes and slowly cooled to room temperature to ensure accurate secondary structure formation. Cells were treated with the aptamers in Krebs-Ringer HEPES buffer [25 mM HEPES (pH 7.4), 120 mM NaCl, 5 mM KCl, 1.2 mM MgSO₄, 1.3 mM CaCl₂), and 1.3 mM KH₂PO₄] for 1 hour.

After aptamer treatment, cells were lysed in lysis buffer [50 mM Tris-HCl (pH 7.4), 150 mM NaCl, 1 mM EDTA, 20 mM NaF, 10 mM β-glycerophosphate, 2 mM Na₃VO₄, 1 mM PMSF, 10% glycerol, 1% Triton-X, and protease inhibitor cocktails]. The cell lysate was centrifuged at 14,000 rpm at 95 °C for 15 minutes to isolate proteins.

The prepared cell lysate was electrophoresed on 6% to 16% SDS-PAGE and transferred to a nitrocellulose membrane. The membrane was reacted with primary antibody for 12 hours at 4 °C, followed by secondary antibody conjugated with IRDye800CW (LI-COR) for 1 hour at room temperature. The presence of protein and the degree of phosphorylation were measured using an infrared fluorescence system (Odyssey, LI-COR).

### 1.2 IGF-1 Receptor Phosphorylation

To determine the effect of the IGF1R-038 aptamer and IGF1R-062 aptamer of Example 1 on the phosphorylation of the IGF-1 receptor, the degree of phosphorylation of the IGF-1 receptor upon treatment with IGF-1, IGF1R-038 aptamer, and IGF1R-062 aptamer was observed using the Western blotting method described above.

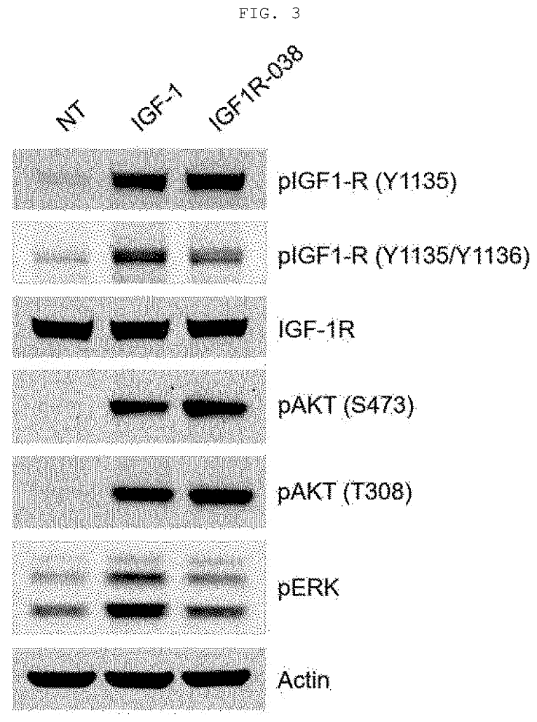

As shown in FIG. 2, phosphorylation at Y1135/Y1136 in the IGF-1 receptor kinase region was found to be increased in both IGF1R-038 and IGF1R-062 aptamer treatment groups.

As confirmed in the results of this experiment, the IGF1R-062 aptamer has one more base added compared to the IGF1R-038 aptamer; however, no difference in activity was observed between the two aptamers, and thus it was judged that the two aptamers are likely to exhibit similar functionality. Therefore, additional experiments were conducted only for the IGF1R-038 aptamer as a representative example.

### 1.3 Phosphorylation of Signaling Proteins

The effect of Y1135 phosphorylation by IGF1R-038 aptamer of Example 1 on IGF-1 signaling was compared with IGF-1.

As shown in FIG. 3, the IGF1R-038 aptamer activated signaling comparable to IGF-1. In 293F cells treated with IGF-1 (10 nM), phosphorylation of the IGF-1 receptor and proteins such as AKT and ERK increased rapidly for 5 minutes and gradually decreased over time. In contrast to IGF-1, IGF1R-038 aptamer (250 nM) slowly increased the phosphorylation of AKT (S473) over 1 hour, and the phosphorylation lasted for 1 hour. In addition, IGF1R-038 aptamer (250 nM) had no effect on phosphorylation of ERK. Thus, the IGF1R-038 aptamer activates signaling of the IGF-1 receptor, but has properties that are distinctly different from IGF-1-induced signaling.

### Experimental Example 2. Evaluation of IGF-1 Receptor Aptamer for IGF-1 Activity Inhibition

Prior to treatment with aptamers, cells were treated with FBS-free DMEM for 3 hours for serum starvation. All aptamers were heated at 95 °C for 5 minutes and slowly cooled to room temperature to ensure accurate secondary structure formation. Cells were treated with 400 nM IGF1R-038 aptamer together with IGF-1 in Krebs-Ringer HEPES buffer [25 mM HEPES (pH 7.4), 120 mM NaCl, 5 mM KCl, 1.2 mM MgSO₄, 1.3 mM CaCl₂), and 1.3 mM KH₂PO₄] for 10 minutes.

After aptamer treatment, cells were lysed in lysis buffer [50 mM Tris-HCl (pH 7.4), 150 mM NaCl, 1 mM EDTA, 20 mM NaF, 10 mM β-glycerophosphate, 2 mM Na₃VO₄, 1 mM PMSF, 10% glycerol, 1% Triton-X, and protease inhibitor cocktails]. The cell lysate was centrifuged at 14,000 rpm at 95 °C for 15 minutes to isolate proteins.

The prepared cell lysate was electrophoresed on 6% to 16% SDS-PAGE and transferred to a nitrocellulose membrane. The membrane was reacted with primary antibody for 12 hours at 4 °C, followed by secondary antibody conjugated with IRDye800CW (LI-COR) for 1 hour at room temperature. The presence of protein and the degree of phosphorylation were measured using an infrared fluorescence system (Odyssey, LI-COR).

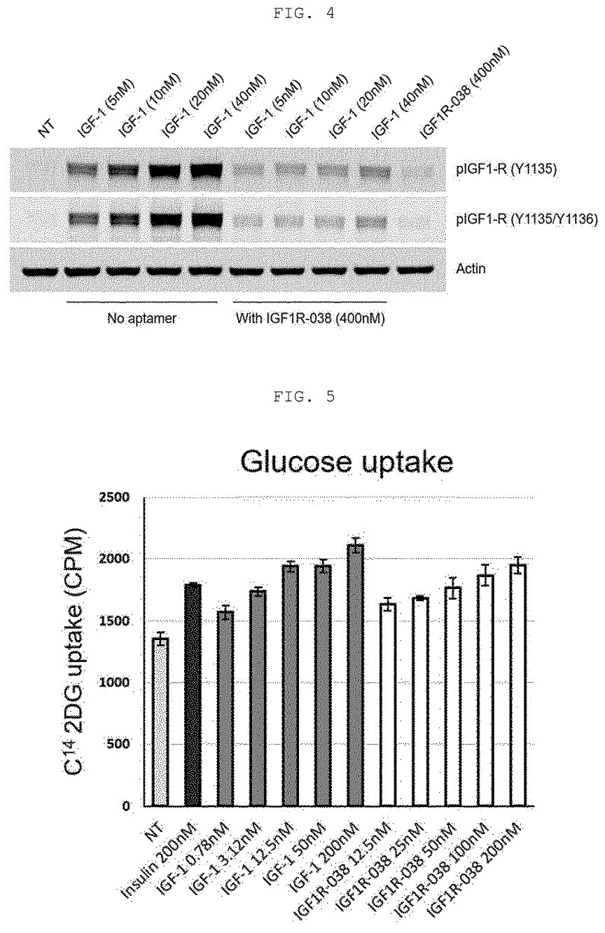

As shown in FIG. 4, it was confirmed that when IGF1R-038 aptamer was mixed with IGF-1 and treated, the effect of IGF-1 was inhibited. This result is considered to be due to the competitive binding of the IGF1R-038 aptamer to IGF-1. These findings indicate that the IGF1R-038 aptamer inhibits IGF-1 activity while retaining its own intrinsic effect, demonstrating that the IGF1R-038 aptamer functions as a biased inhibitor.

### Experimental Example 3. Evaluation of Glucose Uptake by IGF-1 Receptor Aptamer

Since the above Experimental Example 2 showed the IGF1R-038 aptamer increased the phosphorylation of AKT (S473), the activity of the IGF1R-038 aptamer on glucose uptake was tested in L6 myoblast cells.

For this purpose, L6 myoblast cells were treated in DMEM without FBS for 1 hour for serum starvation, and then treated with Krebs-Ringer HEPES buffer solution for 1 hour. The cells were treated with IGF-1 or aptamer at various concentrations (IGF-1: 0.73 nM, 3.12 nM, 12.5 nM, 50 nM, 200 nM; IGF1R-038: 25 nM, 50 nM, 100 nM, 200 nM), followed by treatment with 2-deoxy[14C]glucose (0.1 μCi/ml) for 10 minutes. The cells were washed three times with PBS supplemented with 20 mM glucose and lysed with a solution containing 0.5 N NaOH and 1% SDS. The amount of glucose (2-deoxy-D-glucose) absorbed into the cells was observed using a liquid scintillation counter.

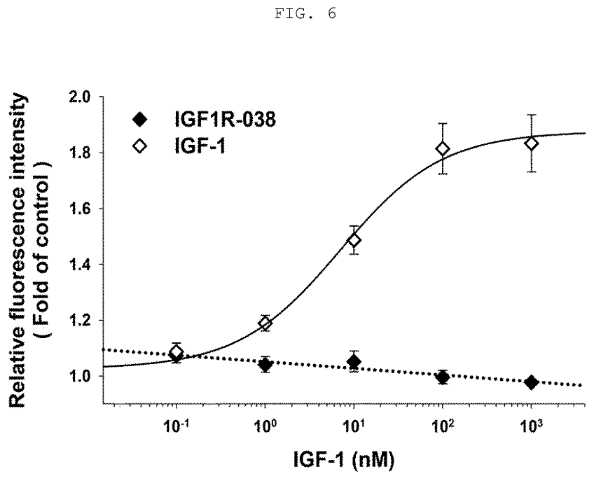

As shown in FIG. 5, the IGF1R-038 aptamer treatment group showed a glucose uptake level comparable to those of the insulin and IGF-1 administration groups. In particular, the IGF1R-038 aptamer treatment group at 100 nM or more showed a higher level of glucose uptake compared to the insulin 200 nM treatment group, indicating that the IGF1R-038 aptamer is suitable for the purpose of lowering blood glucose.

### Experimental Example 4. Experiment on Whether Cancer Cell Growth was Induced

The effect of IGF1R-038 aptamer on cell division was investigated using the MCF-7 cancer cell line, which is widely used to test the ability of IGF-1 receptor to induce cell division

For this purpose, MCF-7 cells were seeded at 10,000 cells per well in a 24-well plate and cultured for 24 hours, followed by an additional 24 hours of culture in 0.5% FBS DMEM for serum starvation. The cells were then treated with IGF-1 and IGF1R-038 alone for 72 hours, with fresh media changes every 24 hours. After treatment, the cells were fixed for 30 minutes at room temperature using PBS containing 4% paraformaldehyde. Then, the cells were quantified by staining their DNA with PBS containing 1 iM SYTO60 fluorescent dye and measuring the fluorescence using a LI-COR Odyssey scanner.

As shown in FIG. 6, treatment of MCF-7 cells with IGF-1 resulted in a 1.85-fold increase in cell division, whereas treatment with IGF1R-038 inhibited cell growth. This means that signaling from IGF1R-038 does not affect the induction of cell division by the IGF-1 receptor. In other words, treatment with the IGF1R-038 aptamer showed therapeutic efficacy without side effects or problems with cell division. In addition, inhibition of cancer cell growth was shown, confirming that the aptamer of the present invention has the potential to be utilized as a cancer therapeutic agent.

1-21. (canceled)

22. An insulin-like growth factor 1 (IGF-1) receptor aptamer that specifically binds to an IGF-1 receptor, comprising SEQ ID NO: 1 or SEQ ID NO: 2.

23. The IGF-1 receptor aptamer of claim 22, wherein the aptamer phosphorylates Y1135 or AKT S473 of the IGF-1 receptor.

24. The IGF-1 receptor aptamer of claim 22, wherein the aptamer consists of SEQ ID NO: 1 or SEQ ID NO: 2.

25. The IGF-1 receptor aptamer of claim 22, wherein the N group of SEQ ID NO: 1 is 5-[N-(1-naphthylmethyl)carboxamide]-2′-deoxyuridine.

26. The IGF-1 receptor aptamer of claim 22, wherein the N group of SEQ ID NO: 2 is 5-[N-(1-naphthylmethyl)carboxamide]-2′-deoxyuridine.

27. The IGF-1 receptor aptamer of claim 22, wherein the sequence of SEQ ID NO: 1 or SEQ ID NO: 2 is SEQ ID NO: 3 or SEQ ID NO: 4.

28. The IGF-1 receptor aptamer of claim 22, wherein the aptamer exists as a dimer or multimer.

29. A method for preventing or treating a metabolic disease or cancer, comprising: administering to a subject in need thereof a therapeutically effective amount of an insulin-like growth factor 1 (IGF-1) receptor aptamer comprising SEQ ID NO: 1 or SEQ ID NO: 2.

30. The method of claim 29, wherein the metabolic disease is any one selected from the group consisting of diabetes, idiopathic type 1 diabetes, latent autoimmune diabetes in adults (LADA), early-onset type 2 diabetes (EOD), youth-onset atypical diabetes (YOAD), maturity-onset diabetes of the young (MODY), malnutrition-related diabetes, gestational diabetes, hyperglycemia, insulin resistance, hepatic insulin resistance, impaired glucose tolerance, diabetic neuropathy, diabetic nephropathy, renal disease, diabetic retinopathy, adipocyte dysfunction, visceral fat accumulation, sleep apnea, obesity, eating disorders, dyslipidemia, and non-alcoholic fatty liver disease (NAFLD).

31. The method of claim 29, wherein the cancer is a cancer with high expression of IGF receptor.

32. The method of claim 29, wherein the cancer is any one selected from the group consisting of Ewing sarcoma, rhabdomyosarcoma, multiple myeloma, lymphoma, leukemia, breast cancer, lung cancer, prostate cancer, pancreatic cancer, rectal cancer, colon cancer, colorectal cancer, cervical cancer, and bile duct cancer.

33. The method of claim 29, wherein the aptamer phosphorylates Y1135 or AKT S473 of the IGF-1 receptor.

34. The method of claim 29, wherein the aptamer consists of SEQ ID NO: 1 or SEQ ID NO: 2.

35. The method of claim 29, wherein the N group of SEQ ID NO: 1 is 5-[N-(1-naphthylmethyl)carboxamide]-2′-deoxyuridine.

36. The method of claim 29, wherein the N group of SEQ ID NO: 2 is 5-[N-(1-naphthylmethyl)carboxamide]-2′-deoxyuridine.

37. The method of claim 29, wherein the sequence of SEQ ID NO: 1 or SEQ ID NO: 2 is SEQ ID NO: 3 or SEQ ID NO: 4.

38. The method of claim 29, wherein the aptamer exists as a dimer or multimer.

39. A pharmaceutical composition for preventing or treating a metabolic disease or cancer, comprising the aptamer according to any one of claim 1; and pharmaceutically acceptable carrier.

40. The pharmaceutical composition of claim 39, wherein the aptamer exists as a dimer or multimer.

41. The pharmaceutical composition of claim 39, wherein the aptamer consists of SEQ ID NO: 1 or SEQ ID NO: 2.
